(19) United States
(12) Patent Application Publication
(10) Pub. No.: US 2007/0025950 A1
(43) Pub. Date: Feb. 1, 2007

(54) COMPOSITION AND METHOD FOR TREATING CELLULITE

(76) Inventor: Melvin L. Elson, Burns, TN (US)

Correspondence Address: Heidi A. Boehlefeld;RENNER, OTTO, BOISSELLE & SKLAR, LLP, 19th Floor, 1621 Euclid Avenue, Cleveland, OH, 44115, US

(21) Appl. No.: 11/459,428

(22) Filed: Jul. 24, 2006

Related U.S. Application Data
(60) Provisional application No. 60/704,196, filed on Jul. 28, 2005.

(51) Int. Cl. A61K 8/97; A61K 36/48; A61K 36/87; A61K 31/12

(52) U.S. Cl. 424/74; 424/757; 424/766; 424/769; 514/682

(57) ABSTRACT

A composition for reducing the appearance of cellulite is provided that comprises guarana extract and vitamin K. In addition, a method of treating cellulite with a topical composition comprising guarana extract and vitamin K is provided.

This application claims priority to provisional patent application Ser. No. 60/704,196 filed Jul. 28, 2005, the content of which is hereby incorporated by reference in its entirety.

### TECHNICAL FIELD

The present invention is directed to a method of treating cellulite and more particularly, to the topical application of a composition for treating cellulite.

### BACKGROUND

Although cellulite was first described in the seventeenth century, the modern era began in the 1970's when the term first began to appear in the lay literature regarding appearance. At that time and somewhat to this day, it was thought that cellulite was merely an accumulation of fatty pockets in the lower aspect of the female body, and that proper diet and exercise could eliminate the problem. Neither the etiology nor the treatment is correct.

Even today, many physicians do not consider cellulite a “real” clinical entity, but merely a normal variant of fat distribution. Considering the myriad of products commercially available to treat and/or prevent cellulite, it is understandable why so much confusion exists as to the nature and treatment of this condition.

For many years, dermatologists and cosmetic surgeons believed that cellulite is just fat deposited and water trapped in women's legs. Now however, the term is beginning to appear more frequently in the medical literature as an accepted clinical entity. Dr. William Coleman III includes a description of cellulite in his textbook Cosmetic Surgery of the Skin: Principles and Techniques, Coleman, William P. III, C. William Hanke III, Thomas H. Alt and Saul Asken, eds., St. Louis, Mo.: Mosby-Year Book, Inc. 1997, in the chapter on liposuction, primarily to emphasize that liposuction is not an acceptable treatment for this condition. He describes cellulite as “a term best applied to the egg carton appearance of the skin of the buttocks and thighs. This dimpled appearance probably represents compartmentalization of the underlying fat between connective tissue fibers that extend from the overlying dermis down to the fascia. This waffling deformity can be seen with or without excessive fat accumulation.” It is of note that this textbook also states that liposuction is not a satisfactory treatment for stretch marks or striae distensae. The association of these two conditions is frequent in the medical and aesthetic lay literature and is important in their etiologies and therapy.

Cellulite, the medical term for which is sclerotic fiberoedema panniculopathy, affects at least 80% and perhaps as many as 95% of post-pubescent females. Neither age nor ageing is a factor in developing the disorder. It is very rare in men, often signaling a problem with low levels of male hormone or increased levels of female hormones. Not all cellulite looks the same, and there is a spectrum of the disorder that can be divided into five grades: Grade I—skin of thighs and buttocks appears normal, but reveals an orange peel effect when pinched; Grade II—orange peel appearance without manipulation; Grade III—horizontal indentations and ripples without compartmentalization; Grade IV—compartmentalization appears in addition to the indentation and ripples with fibrous bands and islands of fat; Grade V—overlapping skin appears in addition to cottage cheese appearance and/or rippling of the tissue.

Obesity may manifest itself in many different ways, but the dimples and wavy appearance with the fibrous bands is present only with cellulite. This is why exercise and diet are totally ineffective as treatments for cellulite. The differences both clinically and pathophysiologically between simple obesity and true cellulite are important. Obesity is simply accumulation of fat cells, which enlarge as well as increase in number in the subcutaneous tissue and internal organs. With cellulite, the individual may be thin, but have quite marked cellulite. This arises from a number of different factors that go into making up a cascade from the influence of estrogen to the final clinical picture.

Estrogen plays a key role in the development of cellulite and without it, cellulite will not occur. Estrogen has major effects on the blood vessels all over the body, particularly noticeable in the skin, such as flushing and rosacea. Estrogen breaks blood vessels, particularly capillaries and it is this damage that starts the cascade to the development of cellulite. Estrogen produces breakage and small vessel deficiency in the subcutaneous tissue just as it does in the skin, and this leads to exudation and edema. Products such as peptides and growth factors begin to accumulate in the tissue due to the vessel damage. In response, lipocytes increase in both numbers and size and with this process, inflammation is induced. Inflammation summons the fibroblast, which is the major cell in wound healing and the formation of collagen and the subsequent fibrous bands.

Regarding the etiology of cellulite, there is vessel weakness and rupture under the influence of estrogen, leading to lipocyte accumulation and fibroblast activation eventuating in the rebuilding and restructuring of the subcutaneous space forming islands of fat surrounded by bridges of fibrosis.

The process is complex and every aspect of the cascade must be addressed for effective treatment. This has not been the case with prior treatments and the direct reason that all previous products and programs have failed. Treatments devised by dermatologists and plastic surgeons generally attempt to break the fibrous bands by, for example, massage, endermologie and laser. However, these treatments are only short-lived. So too are modalities that are used on the skin as they attempt to address the lipocytic problem. These consist mainly of caffeine or retinol and are ineffective in the short or long term. As previously mentioned, diet and exercise have no effect on the appearance of cellulite.

To effectively treat or prevent the occurrence of cellulite, every aspect of the cascade that is initiated by estrogen must be addressed—the vessel damage, the inflammatory process, the lipocyte stimulation and the wound healing.

### SUMMARY

The present invention, in one aspect, is directed to a composition for reducing or eliminating the appearance of cellulite, the composition comprising about 0.001% to about 20% by weight of the composition of guarana extract and about 0.001% to about 15% by weight of vitamin K. In one embodiment, the composition further comprises grape seed extract and licorice extract.

The present invention is further directed to a method of reducing or eliminating the appearance of cellulite comprising topically applying to the skin a composition comprising about 0.001% to about 20% by weight of the composition of guarana extract and about 0.001% to about 15% by weight of vitamin K.

### DETAILED DESCRIPTION

The composition of the present invention comprises, in one embodiment, about 0.001% to about 20% by weight of the composition of guarana extract and about 0.001% to about 15% by weight of vitamin K.

The tropical plant Paullinia cupana (guarana) has been harvested in the Amazon for many years. Treatment of the seeds of the guarana plant and uses thereof in the form of syrups, extracts and distillates as flavoring agents and as a source of caffeine in the soft drink industry are well known.

Guarana seeds contain caffeine (25,000 to 75,000 ppm) as well as trace amounts of theophylline (500 to 750 ppm) and theobromine (300 to 500 ppm). The seeds also contain large quantities of alkaloids, terpenes, tannins, flavinoids, starch, saponins and resinous substances. The xanthine alkaloids (caffeine, theophylline and theobromine) are believed to contribute to guarana's therapeutic activity. The chemicals contained in guarana extract have a lipolytic effect, which aids in the prevention and dissolution of cellulite.

The guarana extract may be present in the composition in an amount of from about 0.001% to about 20% by weight, based on the total weight of the composition. In one embodiment, the guarana extract is present in an amount of from about 0.01% to about 10%, or about 0.05% to about 5% by weight, based on the total weight of the composition.

In addition to the guarana, the composition comprises vitamin K, which is useful in the treatment of blood vessel disorders. Vitamin K is a generic term for a group of substances that contain the 2-methyl-1,4-naphthoquinone ring structure. Vitamin K₁, in addition to being known as phylloquinone, is also known as phytonadione and 2-methyl-3-phytyl-1,4-naphthoqinone. The lipophilic side chain is located at position 3 of the naphthoqinone ring. Its molecular formula is C₃₁H₄₆O₂ and its structural formula is: 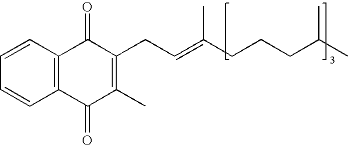
Vitamin K₂ is the collective term for a group of vitamin K compounds called menaquinones. The menaquinone homologues are characterized by the number of isoprene residues comprising the side chain. The side chain is located at position 3 of the naphthoquinone ring. The structural formula for menaquinones is: 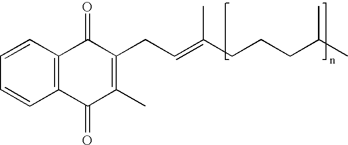
Menaquinones with side chains of up to 15 isoprene units have been described. Menaquinones are designated by the name menaquinone followed by a number. The number refers to the number of isoprene residues in the structure. Thus, menaquinone-4, abbreviated MK-4, possesses four isoprene residues in the side chain. Menaquinone-7 possesses seven isoprene units in the side chain. The menaquinones may also be designated by the number of carbons in the side chain. An isoprene residue contains five carbons. Thus menaquinone-4 is also called vitamin K₂ (20) and menaquinone-7 is also called vitamin K₂ (35). Vitamin K₃ or menadione is a synthetic naphthoqinone derivative. It is also known as 2-methyl-1,4-naphthoqinone. Its molecular formula is C₁₁H₈O₂ and it does not contain a lipophilic side chain.

Vitamin K, particularly, vitamin K₁ has been shown to repair blood vessels, increase absorption of extravascular blood and decrease new vessel formation when applied topically. Vitamin K is the primary mover of the formulation that begins the repair of the mechanism that initiates the development of cellulite, and further prevents the development of cellulite by protecting the vessels from the effects of estrogen.

In one embodiment, the composition comprises about 0.001% to about 15% by weight of vitamin K, based on the total weight of the composition. In one embodiment, the composition comprises about 0.01% to about 10% by weight, or about 0.03% to about 5% by weight, based on the total weight of the composition.

In one embodiment, the composition comprises licorice extract. The licorice extract provides anti-inflammatory properties to the composition, and is believed to promote regeneration of inflammatory tissue. The term “licorice extract” refers to any compound or combination of compounds in the glycyrrhiza family, including glycyrrhiza, glycerrhetic acid (also known as enoxolone, uralenic acid and glycyrrhetinic acid), glycyrrhizic acid (also known as glycyrrhizin, glycyrrhizinic acid and glycyrrhetinic acid glycoside), derivatives thereof, and combinations thereof.

In certain embodiments of the invention, the licorice extract is present in an amount from about 0.00001% to about 10% by weight, based on the total weight of the composition. In one embodiment, the licorice extract is present in an amount of about 0.001% to about 4% by weight of the composition, or about 0.01% to about 3% by weight of the composition.

Other anti-inflammatory agents include one or more of aloe vera, pilewort, Canadian willow root, zinc, arnica, vitamin E, allantoin, chamomile, hydrocortisone, steroids, or non-steroidal anti-inflammatory drugs.

In one embodiment, the composition comprises grape seed extract. Grape seed extract, or vitis vinifera oil, is derived from the red grape seeds and has a high content of oligomeric proanthocyanidins (OPCs), which possess extremely powerful anit-oxidant properties. The anti-oxidant properties improve vascular strength and reduce inflammation, and are much more potent than either vitamin C or E. The OPCs are particularly useful in reducing free radical damage and oxidative stress. The most powerful component of grape seed extract is believed to be the gallic esters of proanthocyanidins, particularly B2-3′-0-gallate, which has the following characteristics: traps hydroxyl free radicals, traps lipid peroxides and free radicals, delays onset of lipid peroxidation, prevents iron-induced peroxidation by binding iron, inhibits production of free radicals and inhibits the damaging effects of enzymes, which can degrade connective tissue structures.

In certain embodiments of the invention, the grape seed extract is present in an amount from about 0.00001% to about 10% by weight, based on the total weight of the composition. In one embodiment, the grape seed extract is present in an amount of about 0.001% to about 3% by weight of the composition, or about 0.01% to about 2% by weight of the composition.

Other anti-oxidant agents include one or more of ascorbic acid (vitamin C) and its salts, ascorbyl esters of fatty acids, ascorbic acid derivatives (e.g., magnesium ascorbyl phosphate), tocopherol (vitamin E), tocopherol sorbate, tocopherol acetate, other esters of tocopherol, hydroxy tyrosol, butylated hydroxy benzoic acids and their salts, 6-hydroxy-2,5,7,8-tetramethylchroman-2-carboxylic acid, gallic acid and its alkyl esters, especially propyl gallate, uric acid and its salts and alkyl esters, sorbic acid and its salts, lipoic acid, amines (e.g., N,N-diethylhydroxylamine, amino-guanidine), sulfhydryl compounds (e.g., glutathione), dihydroxy fumaric acid and its salts, lycine pidolate, arginine pilolate, nordihydroguaiaretic acid, bioflavonoids, lysine, methionine, proline, superoxide dismutase, silymarin, tea extracts, melanin, and rosemary extracts.

The balance of the composition may be a cosmetically or pharmaceutically acceptable delivery system. The composition may be in any suitable form, such as a lotion, cream, gel, spray, thin liquid, mask, serum, solid stick, capsule, salve, mousse and any other cosmetically or pharmaceutically acceptable topical delivery forms. The delivery system can further be traditional water and oil emulsions, suspensions, colloids, microemulsions, clear solutions, suspensions of nanoparticles, emulsions of nanoparticles, or anhydrous compositions.

The cosmetically or pharmaceutically acceptable delivery system or carrier base can optionally include additional ingredients suitable for use in contact with human skin without undue toxicity, incompatibility, instability, allergic response and the like. Non-limiting examples of cosmetic and pharmaceutical ingredients that may be used include skin cleansers, surfactants (cationic, anionic, non-ionic, amphoteric, and zwitterionic), skin conditioning agents, vitamins, hormones, minerals, plant extracts, anti-inflammatory agents, concentrates of plant extracts, emollients, moisturizers, skin protectant, humectants, silicones, skin soothing ingredients, analgesics, skin penetration enhancers, solubilizers, moisturizers, emollients, anesthetics, antibacterial agents, antifungal agents, colorants, perfumes, preservatives, seeds, broken seed nut shells, thickeners, silica, clays, beads, luffa particles, polyethylene balls, mica, pH adjusters, processing aids, fragrances and combinations thereof. The amounts of such ingredients are not limited to any specific numbers, as those versed in this art have learned to utilize only safe, effective, and consumer-preferred amounts of such ingredients and compositions.

The present composition may be used for preventing and/or combating cellulite by topically applying the composition to the affected area. The composition may be applied to the skin weekly, every other day, daily or twice daily. The skin to which the composition is applied is not limited, but particularly includes the skin of the thighs and buttocks. The composition may also be applied to other areas of the body, including the face, and upper arms, to smooth the skin and improve its appearance. The application of the composition to the skin may be continued until the desired degree of improvement is achieved or continued indefinitely for preventative purposes.

### EXAMPLES

### Example 1

A topical lotion is prepared from the following ingredients:

| Ingredient | Amount (Wt. %) |
|---|---|
| Deionized water | 51.80 |
| Propylene glycol | 25.00 |
| SD-Alcohol 40B | 9.00 |
| Triethanolamine | 3.00 |
| Licorice extract | 2.00 |
| Glycerine | 2.00 |
| Grape seed extract | 1.00 |
| Guarana extract | 1.00 |
| Dimethicone | 2.00 |
| Preservative blend* | 1.00 |
| Carbomer | 0.60 |
| Phytonadione | 0.50 |
| Xanthan gum | 0.50 |
| Polysorbate-20 | 0.30 |
| Fragrance | 0.20 |
| Cucumber extract | 0.10 |

*methyl paraben, propyl paraben, diazolidinyl urea

The lotion has a viscosity of 4,500 cstk at 25° C. and a pH of 5.70.

### Example 2

The following ingredients are blended and the resulting mixture is encapsulated. In use, the capsule is broken open and the liquid contained therein is applied to the affected area.

| Ingredient | Amount |
|---|---|
| Cyclopentasiloxane | 10.00-60.00 |
| Dimethicone | 2.00-10.00 |
| C12-15 alkyl benzoate | 2.00-10.00 |
| Grape seed extract | 1.00-2.00 |
| Isopropyl myristate | 0.005-1.00 |
| Paullinia cupana seed (guarana) extract | 0.005-1.00 |
| Phytonadione | 0.005-1.00 |
| Methylmethacrylate/glycol dimethacrylate | 0.005-1.00 |
| Licorice extract | 0.005-1.00 |

Clinical Trial:

A clinical trial was carried out utilizing a topical formula comprising 0.5% by weight of phytonadione, 1.5% by weight of grape seed extract, 0.2% by weight of guarana extract and 0.05% by weight of licorice extract as the active ingredients.

Fourteen women between the ages of 22 and 56 entered the study and agreed to apply a measured amount to each are to be treated (posterior or anterior thighs or buttocks). Two patients dropped out of the study for unknown reasons. Of those completing the study, 3 had stage 11 cellulite, 2 had stage III cellulite, 3 had stage IV cellulite and four had stage V cellulite. All of the patients responded to treatment and none had adverse reactions.

While the invention has been explained in relation to its preferred embodiments, it is to be understood that various modifications thereof will become apparent to those skilled in the art upon reading the specification. Therefore, it is to be understood that the invention disclosed herein is intended to cover such modifications as fall within the scope of the appended claims.

What is claimed is:

1. A method of reducing or eliminating the appearance of cellulite comprising topically applying to the skin in the effected area a composition comprising:
about 0.001% to about 20% by weight of the composition of guarana extract; and
about 0.001% to about 15% by weight of vitamin K.

2. The method of claim 1 wherein the composition further comprises an anti-oxidant agent.

3. The method of claim 2 wherein the anti-oxidant agent comprises grape seed extract.

4. The method of claim 1 wherein the composition further comprises an anti-inflammatory agent.

5. The method of claim 4 wherein the anti-inflammatory agent comprises licorice extract.

6. The method of claim 1 wherein the composition comprises about 0.01 to about 10% by weight guarana extract, based on the total weight of the composition.

7. The method of claim 1 wherein the composition comprises about 0.01 to about 10% by weight vitamin K, based on the total weight of the composition.

8. A method of reducing or eliminating the appearance of cellulite comprising topically applying to the skin in the effected area a composition comprising:
about 0.05% to about 5% by weight of guarana extract;
about 0.03% to about 5% by weight of phytonadione;
about 0.01% to about 3% by weight of licorice extract;
about 0.01% to about 2% by weight of grape seed extract; and
a pharmaceutically acceptable carrier.

9. A composition suitable for topical application comprising:
about 0.001% to about 20% by weight of the composition of guarana; and
about 0.001% to about 15% by weight of vitamin K.

10. The composition of claim 9 further comprising an anti-oxidant agent.

11. The composition of claim 10 wherein the anti-oxidant agent comprises grape seed extract.

12. The composition of claim 9 further comprising an anti-inflammatory agent.

13. The composition of claim 12 wherein the anti-inflammatory agent comprises licorice extract.

14. The composition of claim 9 comprising about 0.01 to about 10% by weight guarana extract, based on the total weight of the composition.

15. The composition of claim 9 comprising about 0.01 to about 10% by weight vitamin K, based on the total weight of the composition.

16. The composition of claim 9 comprising:
about 0.05% to about 5% by weight of guarana extract;
about 0.03% to about 5% by weight of phytonadione;
about 0.01% to about 3% by weight of licorice extract;
about 0.01% to about 2% by weight of grape seed extract; and
a pharmaceutically acceptable carrier.
